(19) United States
(12) Patent Application Publication
(10) Pub. No.: US 2026/0048171 A1
(43) Pub. Date: Feb. 19, 2026

(54) INJECTABLE ACTIVE BONE, PREPARATION METHOD AND USE THEREOF

(71) Applicants: EAST CHINA UNIVERSITY OF SCIENCE AND TECHNOLOGY, Shanghai (CN); Shanghai Reborn Biomaterials Co., Ltd., Shanghai (CN)

(72) Inventors: Yuan YUAN, Shanghai (CN); Zihan WU, Shanghai (CN); Changsheng LIU, Shanghai (CN); Chuanwei ZHOU, Shanghai (CN)

(21) Appl. No.: 19/299,706

(22) Filed: Aug. 14, 2025

(30) Foreign Application Priority Data
Aug. 14, 2024 (CN) 202411112902.4

(51) Int. Cl. A61L 24/04; A61L 24/02; A61L 27/12; A61L 27/26

(52) U.S. Cl. CPC A61L 24/043; A61L 24/02; A61L 27/12; A61L 27/26; A61L 2400/06; A61L 2430/02

(57) ABSTRACT

An injectable bone, a preparation method and use thereof are provided. The injectable bone adhesive has the following components: (c1) an organic component comprising: a first polymer and a second polymer; and (c2) an inorganic component comprising calcium phosphate salts and derivatives thereof. The injectable bone adhesive exhibits outstanding bone adhesion strength and excellent mechanical properties, can closely adhere to complex-shaped bone fragments in comminuted fractures to ensure stable fixation during fracture repair. Additionally, it possesses osteogenic capacity to promote fracture healing.

### CROSS-REFERENCE TO RELATED APPLICATION

This U.S. patent application claims the benefit of and priority to Chinese Application No. 202411112902.4, filed Aug. 14, 2024, the content of which is incorporated herein by reference in its entirety for all purposes.

### TECHNICAL FIELD

The present invention relates to the fields of material science and medicine, specifically to an injectable bone adhesive, preparation method and use thereof.

### BACKGROUND TECHNOLOGY

Fractures, especially comminuted fractures, have been increasing in incidence worldwide, threatening the health and lives of tens of millions of people. Reduction and fixation of bone fragments are crucial methods and procedures for treating comminuted fractures. Currently, metallic internal fixation devices are widely used in clinical practice, but they face notable limitations such as high cost, incompatibility with comminuted fractures, and the need for secondary surgeries. Bone adhesives are a potential alternative product that achieves rapid and secure fixation of fracture sites through adhesion to bone surfaces and the cohesive strength of the material itself, offering advantages such as ease of use, safety, and low cost. However, bone adhesives reported to date generally suffer from deficiencies in mechanical strength and adhesion strength. Meanwhile, minimally invasive surgery is the gold standard in clinical fracture treatment to minimize disruption to the normal physiological environment for bone regeneration, thus requiring bone adhesives with injectable properties.

Therefore, developing an injectable bone adhesive with high mechanical and adhesive strength and certain bone-promoting effects to address clinical needs in fracture repair surgery holds significant research and application value.

### SUMMARY OF THE INVENTION

The objective of the present invention is to provide an injectable bone adhesive with high mechanical and adhesive strength and certain bone-promoting properties, a preparation method and use thereof.

In the first aspect of the present invention, an injectable bone adhesive is provided, wherein the injectable bone adhesive comprises the following components:
(c1) organic component, including a first polymer and a second polymer;
(c2) inorganic component, including a calcium phosphate salt and derivatives thereof.

In another preferred example, the first polymer is a modified polyester polymer, comprising: amino-functionalized polyhydroxyl polyethylene glycolylated polyglyceryl sebacate PEGS-NH₂, amino-functionalized polyglyceryl sebacate, and amino-functionalized polyethylene glycolylated polyglyceryl sebacate.

In another preferred example, the structural unit of the amino-functionalized polyhydroxyl polyethylene glycolylated polyglyceryl sebacate PEGS-NH₂, is shown in Formula I:

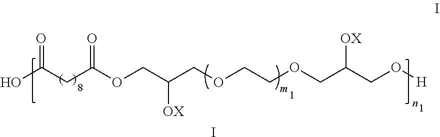

wherein, in formula I, mi is an integer between 1 and 20, and n₁ is an integer between 10 and 80;
each X is independently selected from the group consisting of: H, —CO(CH₂)ₜNH₂, —CO(CH₂)ₜCONHNH₂, and —CO(CH₂)ₜNHC(═NH)NH₂, and on average each structural unit contains 0.5 to 2 —CO(CH₂)ₜNH₂, —CO(CH₂)ₜCONHNH₂, or —CO(CH₂)ₜNHC(═NH)NH₂; wherein t is 1 to 6.

In another preferred embodiment, the first polymer has one or more of the following characteristics:
(a) a number-average molecular weight is 5,000-100,000 Da, preferably 8,000-30,000 Da, more preferably 8,000-18,000 Da, and most preferably 8,000-15,000 Da;
(b) a molecular weight distribution coefficient is 1.0 to 2.0, preferably 1.0 to 1.5;
(c) on average, each structural unit contains at least 0.5 —CO(CH₂)ₜNH₂, —CO(CH₂)ₜCONHNH₂, or —CO(CH₂)ₜNHC(═NH)NH₂, preferably at least 1 —CO(CH₂)ₜNH₂, —CO(CH₂)ₜCONHNH₂ or —CO(CH₂)ₜNHC(═NH)NH₂; wherein t is 1 to 6.

In another preferred embodiment, on average, each structural unit contains at least 1.8 —CO(CH₂)ₜNH₂, —CO(CH₂)ₜCONHNH₂, or —CO(CH₂)ₜNHC(═NH)NH₂; wherein t is 1 to 6.

In another preferred embodiment, the second polymer is a polyamino acid or phosphorylated derivative thereof, selected from the group consisting of: polyglutamic acid, phosphorylated polyglutamic acid PGA-P, polyaspartic acid, and phosphorylated polyaspartic acid.

In another preferred embodiment, the structural unit of the phosphorylated polyglutamic acid PGA-P is shown in Formula II:

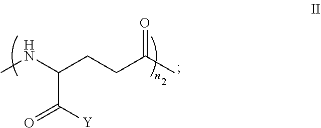

wherein, in formula II, n₂ is an integer between 100 and 2000;
each Y is independently selected from the group consisting of: —OH, —OOP(OH)₂, —NH(CH₂)ₜOOP(OH)₂, and —NH(CH₂)ₜC(OH)(H₂PO₃)₂, and on average each structural unit contains 0.5 to 1 —OOP(OH)₂, —NH(CH₂)ₜOOP(OH)₂ or —NH(CH₂)ₜC(OH)(H₂PO₃)₂; wherein t is 1 to 4.

In another preferred embodiment, the second polymer has one or more of the following characteristics:
(a) a number-average molecular weight is 10-1000 kDa, preferably 10-300 kDa;
(b) a molecular weight distribution coefficient is 1.0 to 2.0, preferably 1.0 to 1.5;
(c) on average, each structural unit contains at least 0.3 —OOP(OH)₂, —NH(CH₂)ₜOOP(OH)₂, or —NH(CH₂)ₜC(OH)(H₂PO₃)₂, preferably at least 0.5 —OOP(OH)₂, —NH(CH₂)ₜOOP(OH)₂, or —NH(CH₂)ₜC(OH)(H₂PO₃)₂; wherein t is 1 to 4.

In another preferred embodiment, on average, each structural unit contains at least 0.7 —OOP(OH)₂, —NH(CH₂)ₜOOP(OH)₂, or —NH(CH₂)ₜC(OH)(H₂PO₃)₂; wherein t is 1 to 4.

In another preferred embodiment, the mass ratio of the first polymer to the second polymer in the organic component is 2:1 to 1:3.

In another preferred embodiment, the mass ratio of the first polymer to the second polymer in the organic component is 1.5:1 to 1:3.

In another preferred embodiment, the calcium phosphate salt and derivatives thereof are selected from the group consisting of: hydroxyapatite, tetracalcium phosphate, tricalcium phosphate, octacalcium phosphate, and calcium dihydrogen phosphate, either alone or in combination of two or more.

In another preferred embodiment, the mass ratio of the organic component to the inorganic component is 1:1 to 4:1.

In another preferred embodiment, the mass ratio of the organic component to the inorganic component is 1:1 to 3:1, preferably 1:1 to 2:1.

In another preferred embodiment, the injectable bone adhesive has one or more of the following characteristics:
(1) High mechanical strength, with a compression modulus of 600-1200 kPa, preferably 800-1000 kPa, and a compression strength of 400-700 kPa, preferably 600-700 kPa;
(2) Excellent elasticity and toughness, with material toughness of 0.1-0.2 MJ·m⁻³, preferably 0.13-0.17 MJ·m³;
(3) Good injectability, with gelation time of 0.3-1 min;
(4) Capable of being injected in situ into the gaps between bone fragments, rapidly curing and strongly adhering to bone tissue.

In the second aspect of the present invention, a method for preparing an injectable bone adhesive as described in the first aspect is provided, the method comprising the steps of:
(a) providing a bone adhesive precursor solution, wherein the bone adhesive precursor solution comprises a first polymer, a second polymer, and calcium phosphate salt;
(b) providing a crosslinking agent solution, wherein the crosslinking agent comprises 1-(3-dimethylaminopropyl)-3-ethylcarbodiimide hydrochloride (EDC) and N-hydroxy succinimide (NHS);
(c) mixing the bone adhesive precursor solution with the crosslinking agent solution to obtain the injectable bone adhesive,
wherein the preparation of the bone adhesive precursor solution includes the following steps:
(a1) dissolving the first polymer and the second polymer in a solvent to obtain a mixed solution;
(a2) adding the calcium phosphate salt to the above mixed solution to obtain the bone adhesive precursor solution.

In another preferred example, the first polymer and the second polymer are crosslinked via amide bond.

In another preferred example, the step (c) comprises: transferring a mixture of the bone adhesive precursor solution and the crosslinking agent solution into a syringe, and injecting to form the bone adhesive.

In another preferred embodiment, the crosslinking agent in step (b) is 1-(3-dimethylaminopropyl)-3-ethylcarbodiimide hydrochloride (EDC) and N-hydroxy succinimide (NHS) for catalyzing the formation of amide bonds.

In another preferred example, the molar ratio of EDC to NHS in the crosslinking agent described in step (b) is 0.5-2:1, and the molar ratio of EDC to the amino groups in the first polymer is 0.5-1:1.

In another preferred example, the solvent in step (a1) is selected from: ultrapure water, PBS solution (pH=7.2), or a combination thereof.

In another preferred embodiment, the viscosity of the bone adhesive precursor solution is 80-120 mPa-s.

In another preferred embodiment, the molar ratio of 1-(3-dimethylaminopropyl)-3-ethylcarbodiimide hydrochloride to N-hydroxy succinimide is 0.5-2:1, preferably 0.8-1.2:1, and more preferably 1:1.

In another preferred embodiment, the concentration of 1-(3-dimethylaminopropyl)-3-ethylcarbodiimide hydrochloride in the crosslinking agent is 0.2-0.7 g/mL.

In another preferred embodiment, the viscosity of the crosslinking agent solution is 0.6-0.8 mPa·s.

In another preferred embodiment, the injectable bone adhesive exhibits excellent mechanical properties and bone tissue adhesion properties.

In another preferred embodiment, the method for preparing the first polymer comprises the following steps:
(a1) adding sebacic acid and tetrabutylammonium hydroxide to anhydrous ethanol at a molar ratio of 1:2-8, followed by stirring to obtain a white product;
(b1) dissolving sebacic acid, polyethylene glycol diglycidyl ether (PEGDGE), and the white product obtained in step (a1) in anhydrous N,N-dimethylformamide at a molar ratio of 1:0.8-1.2:0.003-0.009 under a nitrogen atmosphere, followed by stirring at 90-120° C. for 60-96 hours to obtain a first mixture;
(c1) dialyzing and purifying the first mixture obtained in step (b1) to obtain purified polyethylene glycol-polyglyceryl sebacate;
(d1) dissolving the purified polyethylene glycol-polyglyceryl sebacate (calculated by hydroxyl content) obtained from step (c1), the amination reagent, N, N′-diisopropylcarbodiimide (DIC), and 4-dimethylaminopyridine (DMAP) in anhydrous dichloromethane at a molar ratio of 1:1-1.4:1.5-2.5:0.02-0.07 under nitrogen atmosphere protection, followed by stirring at room temperature for 20-30 hours to obtain a second mixture;
(e1) filtering the second mixture obtained in step (d1) under vacuum, concentrating, and then treating with trifluoroacetic acid to remove the tert-butoxycarbonyl (Boc), followed by dialyzing and purifying to yield the first polymer.

The amination reagent is selected from the group consisting of: N-(tert-butoxycarbonyl)glycine (BOC-glycine), N-(tert-butoxycarbonyl)aminovaleric acid, acylhydrazide acetic acid, and guanidinoacetic acid.

In another preferred embodiment, the method for preparing the second polymer comprises the following steps:
(a2) dissolving the polyamino acid, phosphorylation reagent, and 1-(3-dimethylaminopropyl)-3-ethylcarbodiimide hydrochloride (EDC) and N-hydroxy succinimide (NHS) in ultrapure water at a molar ratio of 1:0.8-1.2:1.2-1.8:1.2-1.8, followed by stirring at room temperature for 20-30 hours to obtain a colorless transparent liquid;
(b2) dialyzing and purifying the colorless transparent liquid obtained in step (a2) and lyophilizing to yield the second polymer.

In another preferred embodiment, the polyamino acid is a polyamino acid having side carboxyl groups.

In another preferred embodiment, the polyamino acid is selected from the group consisting of: polyglutamic acid, polyaspartic acid, and partial short peptides.

In another preferred embodiment, the short peptide is glutathione and RGD peptide.

In another preferred embodiment, the phosphorylation agent is selected from the group consisting of: O-phosphoethanolamine, pamidronic acid, and alendronic acid.

In another preferred embodiment, the mass ratio of the first polymer to the second polymer is 2:1 to 1:3, preferably 1:1 to 1:2, and more preferably 1:1 to 1:1.5.

In another preferred embodiment, the concentration of the first polymer in step (a1) is 20-50%, and the concentration of the second polymer is 40-70%, calculated as mass-volume percentage of solute (g)/solvent (mL).

In the third aspect of the present invention, a kit is provided for forming the injectable bone adhesive described in the first aspect, wherein the kit comprises: the bone adhesive precursor solution and the crosslinking agent solution described in the second aspect.

In another preferred embodiment, the bone adhesive precursor solution and crosslinking agent solution are mixed to form a mixture, wherein the initial viscosity of the mixture is 80-120 mPa-s, meeting the injectable requirements.

In another preferred embodiment, the mixture is injected into a target bone tissue site requiring adhesion and undergoes in situ crosslinking to form the bone adhesive according to the first aspect of the present invention.

In another preferred embodiment, the gelation time of the mixture is 0.3-1 minute.

In the fourth aspect of the present invention, use of the injectable bone adhesive according to the first aspect is provided, for any one or two or more of the following applications:
(i) preparation of a bone fragment bonding material for fracture repair surgery;
(ii) preparation of a bone tissue defect filler material;
(iii) preparation of a guided tissue regeneration material;
(iv) preparation of a medical cosmetology material.

In another preferred embodiment, a medical glue is provided, wherein the medical glue comprises the injectable bone adhesive described in the first aspect.

In another preferred embodiment, a medical material is provided, wherein the medical material comprises the injectable bone adhesive described in the first aspect.

In another preferred embodiment, the medical material is selected from the group consisting of: medical bone tissue adhesive, medical dental tissue adhesive, medical adhesive, medical bone repair material, and medical dental repair material.

In another preferred embodiment, a method for filling bone tissue defects and/or guiding bone tissue regeneration is provided, the method comprising applying the injectable bone adhesive described in the first aspect to the site to be treated.

It should be understood that, within the scope of the present invention, all aforementioned technical features of the invention and those specifically described in the following sections (e.g., examples) may be mutually combined to form new or preferred technical solutions. For conciseness, not all possible combinations are exhaustively described herein.

### DESCRIPTION OF THE DRAWINGS

FIG. 1 shows a schematic diagram of the structure and application of the bone adhesive material in the present invention.

FIG. 2 shows the nuclear magnetic resonance hydrogen spectrum (A) and Fourier transform infrared spectrum (B) of the amino-functionalized polyethylene glycolylated polyglyceryl sebacate in the present invention.

FIG. 3 shows the nuclear magnetic resonance hydrogen spectrum (A), nuclear magnetic resonance phosphorus spectrum (B), and Fourier transform infrared spectrum (C) of the phosphorylated polyglutamic acid in the present invention.

FIG. 4 shows the injectability of the injectable bone adhesive of the present invention.

FIG. 5 shows the time-scan rheological curve of the injectable bone adhesive of the present invention.

FIG. 6 shows the effect of different formulations on the wet environment bonding performance of the injectable bone adhesive. The effect of different organic-inorganic ratios on the bonding strength of the injectable bone adhesive (A); the effect of different ratios of the first polymer and second polymer in the organic component on the bonding strength of the injectable bone adhesive (B).

FIG. 7 shows the wet environment bonding curve diagram (A) and bonding strength diagram (B) of the injectable bone adhesive of the present invention at different crosslinking degrees. The p-value is a numerical value indicating the level of statistical significance, and a value less than 0.05 indicates a significant difference between the two groups of data, where *p<0.05, **p<0.01, ***p<0.001, ****p<0.0001.

FIG. 8 shows the wet environment compression stress-strain curve diagram (A), compression modulus diagram (B), and compression strength diagram (C) of the injectable bone adhesive of the present invention. The p-value is a numerical value indicating the level of statistical significance, and a value less than 0.05 indicates a significant difference between the two sets of data, where *p<0.05, **p<0.01, ***p<0.001, ****p<0.0001.

FIG. 9 shows the compressive stress-strain curve diagram (A) and energy loss diagram (B) of the injectable bone adhesive in the present invention under cyclic loading in a wet environment. The p-value is a numerical value indicating the level of statistical significance, and a value less than 0.05 indicates a significant difference between the two sets of data, where *p<0.05, **p<0.01, ***p<0.001, ****p<0.0001.

FIG. 10 shows a comparison of the swelling and mechanical properties of adhesives from different groups in the present invention. It demonstrates the comparison of the presence or absence of chemical cross-linking, the presence or absence of calcium phosphate reinforcement, and the strength of electrostatic interactions between phosphate groups and carboxyl groups. Actual images of different groups after 24 hours of swelling (A); swelling rates of different groups after 24 hours of swelling (B); compression modulus diagrams of different groups after 24 hours of swelling (C); compression strength of different groups after swelling for 24 hours (D). The p-value is a numerical value indicating the level of statistical significance, and a value less than 0.05 indicates a significant difference between the two groups of data, where *p<0.05, **p<0.01, ***p<0.001, ****p<0.0001.

FIG. 11 shows the cellular safety of the injectable bone adhesive of the present invention. The p-value is a numerical value indicating the level of statistical significance, and a value less than 0.05 indicates a significant difference between the two groups of data, where *p<0.05, **p<0.01, ***p<0.001, ****p<0.0001.

FIG. 12 shows the performance of the injectable bone adhesive in the present invention in promoting osteogenic differentiation of mesenchymal stem cells.

FIG. 13 shows CT images of tibial fracture fragment bonding repair at 4 and 8 weeks using the injectable bone adhesive of the present invention.

### DETAILED DESCRIPTION OF THE INVENTION

Through extensive and in-depth research with rigorous experimental screening, the inventors have unexpectedly developed for the first time an injectable bone adhesive exhibiting high mechanical/adhesive strength and osteogenic activity for bone fragment bonding in fracture repair surgeries. In the present invention, amino-functionalized polyhydroxyl polyethylene glycolylated polyglyceryl sebacate (PEGS-NH₂>) and phosphorylated polyglutamic acid (PGA-P) are used as the primary matrix materials, combined with tetracalcium phosphate (TTCP). The electrostatic interaction between phosphate groups and carboxyl groups with calcium ions yields a bone adhesive precursor solution with a certain viscosity. Ultimately, under the action of EDC/NHS crosslinkers, the precursor solution can be mixed and injected onto bone tissue surfaces, then rapidly cured in situ to establish robust adhesion.

The injectable bone adhesive of the present invention has a dual cross-linked network internally, including covalent chemical cross-linking formed by PEGS-NH₂ and PGA-P in the organic component under the action of the cross-linking agent, and electrostatic physical cross-linking spontaneously formed between PGA-P and inorganic calcium phosphate salts. Additionally, the injectable bone adhesive of the present invention can form various bonds with the surface of bone tissue, including stable amide covalent bonds, electrostatic complexation between phosphate groups and carboxyl groups with calcium ions, and hydrogen bonding. The introduction of calcium phosphate salt components not only enhances the mechanical properties of the bone adhesive but also improves its osteoconductivity, promoting new bone regeneration and facilitating fracture healing. Furthermore, it exhibits excellent injectability, enabling it to be injected onto the bone tissue surface, where it cures in situ to form a gel and establishes strong adhesive bonds. Therefore, the injectable bone adhesive of the present invention can be used for bonding bone fragments in fracture repair surgery, demonstrating clinical application potential. The hydrogel of the present invention possesses superior mechanical properties and osteogenic-promoting performance. Based on this, the present invention was developed.

### Terms

Unless otherwise defined, all technical and scientific terms used herein have the same meanings as commonly understood by one of ordinary skill in the art to which this invention belongs.

When used in reference to a specifically enumerated numerical value, the term “about” means the value may vary from the stated value by no more than 1%. For example, the expression “about 100” encompasses all values between 99 and 101 (e.g., 99.1, 99.2, 99.3, 99.4, etc.).

As used herein, the terms “comprising” or “including” may be open-ended, partially closed, or closed. In other words, these terms also encompass “consisting essentially of” or “consisting of”.

The term “bone adhesive precursor solution” refers to a homogeneous dispersion formed by dissolving components for constituting a bone adhesive (including, but not limited to, a first polymer, a second polymer, and a calcium phosphate salt) in a specific solvent.

The term “crosslinking agent” refers to a component capable of crosslinking constituents in the bone adhesive precursor solution, thereby forming a bone adhesive with a three-dimensional spatial structure.

For long-chain polymers, the term “dispersity index” refers to the ratio of weight-average molecular weight (Mw) to number-average molecular weight (Mn) for long-chain polymers, expressed as D=Mw/Mn, where D≥1. Due to the polydispersity of polymers, their mass distribution is non-uniform. The dispersity index characterizes the mass distribution of polymer products, with values closer to 1 indicating a more uniform mass distribution.

The terms “amino-functionalized polyhydroxyl polyethylene glycolylated polyglyceryl sebacate “amino-functionalized PEGS”, and “PEGS-NH₂” are used interchangeably herein.

The terms “phosphorylated polyglutamic acid” and “PGA-P” are used interchangeably herein.

The terms “1-(3-dimethylaminopropyl)-3-ethylcarbodiimide hydrochloride” and “EDC” are used interchangeably herein.

The terms “N-hydroxy succinimide” and “NHS” are used interchangeably herein.

The terms “phosphate-buffered saline solution” and “PBS” are used interchangeably herein.

The terms “bis(tetrabutylammonium hydroxide) sebacate” and “TBAS” are used interchangeably herein.

The terms “polyethylene glycol diglycidyl ether” and “PEGDGE” are used interchangeably herein.

The terms “N-(tert-butoxycarbonyl)glycine” and “BOC-glycine” are used interchangeably herein.

The terms “N,N′-diisopropylcarbodiimide” and “DIC” are used interchangeably herein.

The terms “4-dimethylaminopyridine” and “DMAP” are used interchangeably herein.

As used herein, in the terms “NPC4”, “NPC5”, “NPC6”, and “NPC7”, N represents PEGS-NH₂, P represents PGA-P, C represents calcium phosphate salt, and 4, 5, 6, and 7 indicate the crosslinking degree of the injectable bone adhesive.

### The Injectable Bone Adhesive of the Present Invention

The bone adhesive of the present invention is an organic-inorganic composite structure composed of biomimetic bone, wherein the organic component includes a first polymer and a second polymer, and the inorganic component includes calcium phosphate and its derivatives. The bone adhesive is prepared by crosslinking the first polymer with the second polymer and compositing with calcium phosphate salt.

Specifically, the first polymer is a modified polyester polymer, selected from the group consisting of: amino-functionalized polyhydroxyl polyethylene glycolylated polyglyceryl sebacate PEGS-NH₂, amino-functionalized polyglyceryl sebacate, and amino-functionalized polyethylene glycolylated polyglyceryl sebacate.

In a preferred embodiment of the present invention, the structural unit of the amino-functionalized polyhydroxyl polyethylene glycolylated polyglyceryl sebacate PEGS-NH₂, is shown in Formula I:

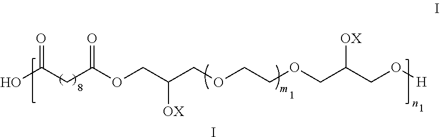

wherein, in formula I, mi is an integer between 1 and 20, and n₁ is an integer between 10 and 80;
each X is independently selected from the group consisting of: H, —CO(CH₂)ₜNH₂, —CO(CH₂)ₜCONHNH₂, and —CO(CH₂)ₜNHC(═NH)NH₂, and on average each structural unit contains 0.5 to 2 —CO(CH₂)ₜNH₂, —CO(CH₂)ₜCONHNH₂, or —CO(CH₂)ₜNHC(═NH)NH₂; wherein t is 1 to 6.

Preferably, the first polymer has one or more of the following characteristics:
(a) a number-average molecular weight is 5,000-100,000 Da, preferably 8,000-30,000 Da, more preferably 8,000-18,000 Da, and most preferably 8,000-15,000 Da;
(b) a molecular weight distribution coefficient is 1.0 to 2.0, preferably 1.0 to 1.5;
(c) on average, each structural unit contains at least 0.5 —CO(CH₂)ₜNH₂, —CO(CH₂)ₜCONHNH₂, or —CO(CH₂)ₜNHC(═NH)NH₂, preferably at least 1 —CO(CH₂)ₜNH₂, —CO(CH₂)ₜCONHNH₂, or —CO(CH₂)ₜNHC(═NH)NH₂; wherein t is 1 to 6.

Preferably, on average, each structural unit contains at least 1.8 —CO(CH₂)ₜNH₂, —CO(CH₂)ₜCONHNH₂, or —CO(CH₂)ₜNHC(═NH)NH₂; wherein t is 1 to 6.

In a preferred embodiment of the present invention, the second polymer is a polyamino acid or its phosphorylated derivative, including polyglutamic acid, phosphorylated polyglutamic acid PGA-P, polyaspartic acid, and phosphorylated polyaspartic acid.

Preferably, the structural unit of the phosphorylated polyglutamic acid PGA-P is shown in Formula II:

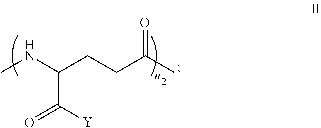

wherein, in formula II, n₂ is an integer between 100 and 2000;
each Y is independently selected from the group consisting of: —OH, —OOP(OH)₂, —NH(CH₂)ₜOOP(OH)₂, and —NH(CH₂)ₜC(OH)(H₂PO₃)₂, and on average each structural unit contains 0.5 to 1 —OOP(OH)₂, —NH(CH₂)ₜOOP(OH)₂, or —NH(CH₂)ₜC(OH)(H₂PO₃)₂; wherein t is 1 to 4.

Preferably, the second polymer has one or more of the following characteristics:
(a) a number-average molecular weight is 10-1000 kDa, preferably 10-300 kDa;
(b) a molecular weight distribution coefficient is 1.0 to 2.0, preferably 1.0 to 1.5;
(c) on average, each structural unit contains at least 0.3 —OOP(OH)₂, —NH(CH₂)ₜOOP(OH)₂, or —NH(CH₂)ₜC(OH)(H₂PO₃)₂, preferably at least 0.5 OOP(OH)₂, —NH(CH₂)ₜOOP(OH)₂, or —NH(CH₂)ₜC(OH)(H₂PO₃)₂; wherein t is 1 to 4.

Preferably, on average, each structural unit contains at least 0.7 —OOP(OH)₂, —NH(CH₂)ₜOOP(OH)₂, or —NH(CH₂)ₜC(OH)(H₂PO₃)₂; wherein t is 1 to 4.

In a preferred embodiment of the present invention, the mass ratio of the first polymer to the second polymer in the organic component is 2:1 to 1:3.

Preferably, the mass ratio of the first polymer to the second polymer in the organic component is 1.5:1 to 1:3.

In a preferred embodiment of the present invention, the calcium phosphate salt and derivatives thereof are selected from the group consisting of: hydroxyapatite, tetracalcium phosphate, tricalcium phosphate, octacalcium phosphate, and calcium dihydrogen phosphate, either alone or in combination of two or more.

In a preferred embodiment of the present invention, the mass ratio of the organic component to the inorganic component is 1:1 to 4:1.

Preferably, the mass ratio of the organic component to the inorganic component is 1:1 to 3:1, preferably 1:1 to 2:1.

Preferably, the injectable bone adhesive has one or more of the following characteristics:
(1) high mechanical strength, with a compression modulus of 600-1200 kPa, preferably 800-1000 kPa, and a compressive strength of 400-700 kPa, preferably 600-700 kPa;
(2) excellent elasticity and toughness, with material toughness of 0.1-0.2 MJ·m⁻³, preferably 0.13-0.17 MJ·m⁻³;
(3) good injectability, with gelation time of 0.3-1 min;
(4) capable of being injected in situ into bone fragment gaps, rapidly curing and strongly adhering to bone tissue.

The biocompatible bone adhesive of the present invention exhibits excellent injectability and an optimal curing time, along with superior topological adaptability that enable it to closely adhere to complex-shaped bone fragments in comminuted fractures, thereby enhancing adhesion strength and regulating osteogenic microenvironment. The adhesive demonstrates outstanding bone adhesion strength and good mechanical properties, ensuring stable fixation of bone fragments during fracture repair. Furthermore, it possesses osteogenic capacity to promote fracture healing. The bone adhesive of the present invention is a clinically promising material for bonding bone fragment in fracture treatment.

Compared with the prior art, the main advantages of the present invention are as follows:
1) The bone adhesive of the present invention exhibits outstanding mechanical properties and adhesion strength;
2) The bone adhesive of the present invention demonstrates excellent toughness and fatigue resistance;
3) The bone adhesive of the present invention can be injected to the desired sites for in situ curing, with adjustable curing time between 0.3-1 minutes;
4) The bone adhesive of the present invention possesses superior topological properties, enabling it to closely conform to the shape of bone fragments;
5) The rheological properties, injectability, mechanical properties and cellular behaviour of the bone adhesive of the present invention can be precisely regulated and optimized by modifying the content of its components;
6) The bone adhesive of the present invention is biodegradable with a degradation time that matches the growth of new bone;
7) The bone adhesive of the present invention has the ability to promote bone regeneration and the growth of new bone;
8) The minimally invasive nature of the bone adhesive makes it highly promising for clinical applications.

The present invention is further illustrated by the following specific examples. It should be understood that these examples are provided for illustrative purposes only and are not intended to limit the scope of the invention. For experimental methods where specific conditions are not indicated in the examples below, they are generally carried out under conventional conditions or according to the manufacturer's recommended conditions. Unless otherwise stated, all percentages and parts are expressed by weight.

### Example 1. Preparation of Bone Tissue Adhesive Hydrogel

### 1.1 Synthesis and Purification of PEGS-NH₂

### 1.1.1 N-(Tert-Butoxycarbonyl)Glycine Modified PEGS

(a1) Sebacic acid (1.95 g, 0.01 mol) and tetrabutylammonium hydroxide (20 g of 25 wt % aqueous solution, 0.07 mol) were added to 100 mL of 95% ethanol. The reaction mixture was stirred at 55° C. for 30 min, followed by rotary evaporation at 40° C. to remove ethanol. The product, bis(tetrabutylammonium hydroxide) sebacate (TBAS), was obtained by lyophilization.

(b1) Under nitrogen atmosphere, sebacic acid (20.23 g, 0.1 mol), poly(ethylene glycol) diglycidyl ether (50.00 g), and TBAS from step (a1) (0.41 g) were dissolved in 180 mL anhydrous N,N-dimethylformamide and stirred at 100° C. for 72 h;

(c1) The product from step (b1) was purified by dialysis to obtain purified linear polyester PEGS;

(d1) Under nitrogen protection, linear polyester PEGS from step (c1) (10.53 g), N-(tert-butoxycarbonyl)glycine (6.31 g), N,N′-diisopropylcarbodiimide (7.57 g, 0.06 mol), and 4-dimethylaminopyridine (0.18 g, 0.001 mol) were dissolved in 150 mL anhydrous dichloromethane, and stirred at room temperature for 24 h;

(e1) The product from step (d1) was vacuum-filtered, concentrated, and treated with trifluoroacetic acid to remove the tert-butoxycarbonyl (Boc) protecting group. The amino-functionalized polyester PEGS (PEGS-NH₂) was obtained by dialysis purification using a membrane with 3,500 Da molecular weight cutoff.

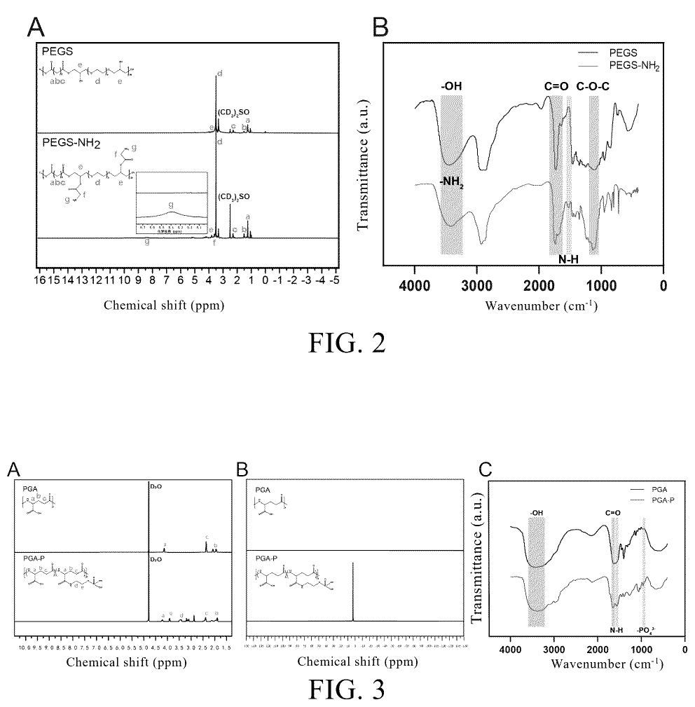

The obtained amino-functionalized polyester PEGS appears as a pale yellow transparent viscous substance, with a molecular weight of 8,000-18,000 Da, a molecular weight distribution coefficient of 1.20-1.50, and an actual amino grafting rate of 90-95%. The ¹H NMR spectrum and Fourier transform infrared (FTIR) spectrum are shown in FIG. 2 (panels A and B).

To further demonstrate the diversity and feasibility of amino-functionalized PEGS synthesis, N-(tert-butoxycarbonyl) glycine in the above method was replaced with N-(tert-butoxycarbonyl)aminovaleric acid, acylhydrazide acetic acid, and guanidinoacetic acid, respectively, and successful amino-functionalized modification of PEGS was also achieved.

1.1.2 Modification of PEGS with N-(Tert-Butoxycarbonyl)Aminovaleric Acid

In this experiment, the preparation method was the same as that in 1.1.1, except that N-(tert-butoxycarbonyl)aminovaleric acid was used instead of N-(tert-butoxycarbonyl)glycine for the amination of PEGS. The results showed that the obtained amino-functionalized polyester PEGS appeared as a yellow transparent viscous substance, with a molecular weight of 8,000-20,000 Da, a molecular weight distribution coefficient of 1.2-1.5, and an actual amino grafting rate of 85-95%.

### 1.2 Synthesis and Purification of PGA-P

### 1.2.1 O-Phosphoethanolamine-Modified PGA

(a2) Polyglutamic acid (PGA), O-phosphorylethanolamine, 1-(3-dimethylaminopropyl)-3-ethylcarbodiimide hydrochloride (EDC), and N-hydroxysuccinimide (NHS) were dissolved in ultrapure water at a molar ratio of 1:1:1.5:1.5. The reaction mixture was stirred at room temperature for 24 h to obtain a colorless transparent solution.

(b2) The solution from step (a2) was purified by dialysis and lyophilized to yield phosphorylated polyglutamic acid (PGA-P).

The resulting phosphorylated polyglutamic acid appears as white floccule, with a molecular weight of 300-400 kDa, a molecular weight distribution coefficient of 1.20-1.50, and an actual phosphate group grafting rate of 70-80%. The nuclear magnetic resonance hydrogen spectrum, phosphorus spectrum, and Fourier transform infrared spectrum are shown in FIGS. 3, panels A, B, and C, respectively.

To further demonstrate the versatility and feasibility of this phosphorylation approach, we used pamidronic acid and alendronic acid instead of O-phosphorylethanolamine and also successfully modified PGA.

### 1.2.2 Pamidronic Acid-Modified PGA

The synthesis procedure in this experiment followed the same protocol as described in 1.2.1, with the exception that pamidonic acid was used instead of O-phosphoethanolamine.

The resulting phosphorylated polyglutamic acid (PGA) exhibits a white, fluffy appearance, with a molecular weight ranging from 300 to 500 kDa, a molecular weight distribution coefficient of 1.2 to 1.5, and an actual phosphate group grafting rate of 50 to 70%.

### 1.3 Preparation of Injectable Bone Adhesive

The specific formulation was shown in the table below. The preparation method of the tissue-adhesive hydrogel comprised the following steps:

(a3) Dissolve PEGS-NH₂ and PGA-P in ultrapure water and stir evenly.

(b3) Add tetracalcium phosphate (TTCP) into the solution obtained from step (a3) and stir evenly.

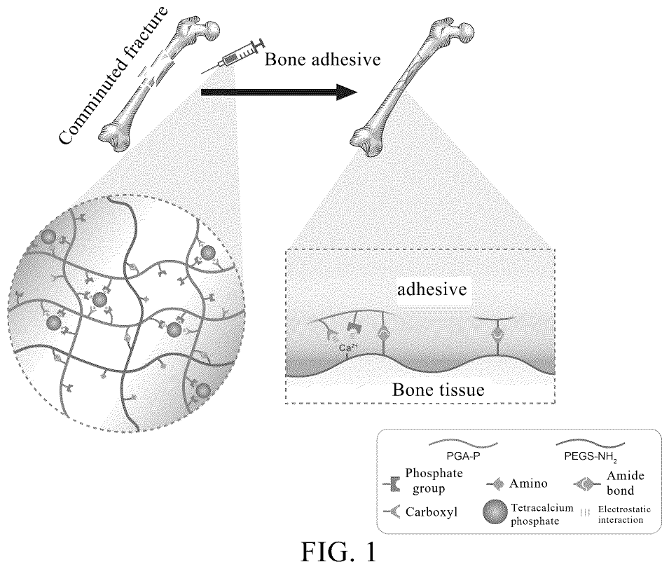

(c3) Add an EDC/NHS aqueous solution into the mixture from step (b3), stir quickly and evenly to form an injectable gel. FIG. 1 shows a schematic diagram of the structure and application of the bone adhesive material of the present invention.

TABLE 1
Formulation ratios of raw materials for preparing injectable
bone adhesives with different crosslinking degrees

Sample

| No. | TTCP | PGA-P | PEGS-NH₂ | EDC | | NHS |
|---|---|---|---|---|---|---|
| NPC0 | 45 mg | 50 mg | 40 mg | 0 | | 0 |
| NPC5 | 45 mg | 50 mg | 40 mg | 9.38 | mg | 5.63 mg |
| NPC6 | | | | 11.25 | mg | 6.75 mg |
| NPC7 | | | | 13.13 | mg | 7.88 mg |
| NPC8 | | | | 15.00 | mg | 9.00 mg |

TABLE 2
Different organic/inorganic ratios

| Sample | | | | | |
|---|---|---|---|---|---|
| No. | TTCP | PGA-P | PEGS-NH₂ | EDC | NHS |
| NPC30 | 30 mg | 50 mg | 30 mg | 8.44 mg | 5.06 mg |
| NPC40 | 40 mg | | | | |
| NPC45 | 45 mg | | | | |
| NPC50 | 50 mg | | | | |
| NPC60 | 60 mg | | | | |

TABLE 3
Different proportions of organic components

Sample

| No. | TTCP | PGA-P | PEGS-NH₂ | EDC | | NHS |
|---|---|---|---|---|---|---|
| N2PC | 45 mg | 50 mg | 20 mg | 5.63 | mg | 3.38 mg |
| N3PC | | | 30 mg | 8.44 | mg | 5.06 mg |
| N4PC | | | 40 mg | 11.25 | mg | 6.75 mg |
| N5PC | | | 50 mg | 14.06 | mg | 8.44 mg |

TABLE 4
Mechanism Investigation (Chemical vs. Physical Crosslinking)

| Sample | | | | | | |
|---|---|---|---|---|---|---|
| No. | TTCP | PGA-P | PGA | PEGS-NH₂ | EDC | NHS |
| NPC 6 | 45 mg | 50 mg | 0 | 40 mg | 11.25 mg | 6.75 mg |
| NAC | 45 mg | 0 | 50 mg | | 11.25 mg | 6.75 mg |
| NP | 0 | 50 mg | 0 | | 11.25 mg | 6.75 mg |
| NPC 0 | 45 mg | 50 mg | 0 | | 0 | 0 |

Through electrostatic interactions, the bone adhesive precursor solution forms a flowable, stable, and homogeneously dispersed composite. Under the action of crosslinking agents EDC/NHS, chemical crosslinks form between PEGS-NH₂ and PGA-P. In this example, five bone adhesives with varying crosslinking degrees were prepared by adjusting the molar ratio of EDC/NHS to amino groups in amino-functionalized PEGS (0-70%), as shown in Table 1. Table 2 presents bone adhesives prepared with different organic/inorganic ratios, while Table 3 shows adhesives obtained with varying proportions of organic components. To investigate the adhesive mechanism, crosslinking mechanisms were studied (Table 4). Notably, the 0% content represents bone adhesive hydrogel without chemical crosslinking (no EDC/NHS added).

To further demonstrate the modifiability of PGA, this experiment replaced PGA with polyaspartic acid—another amino acid polymer containing side-chain carboxyl groups—to prepare bone adhesives using identical formulation ratios. Experimental results revealed that the bone adhesive prepared with phosphorylated polyaspartic acid exhibited similar physicochemical property trends to the hydrogel produced with PGA-P.

### 1.4 Preparation of Bone Adhesive—Polyaspartic Acid

In this experiment, phosphorylated polyaspartic acid was synthesized following the same procedure described in 1.2, with the exception that PGA was replaced with polyaspartic acid (another carboxyl-containing amino acid polymer).

The preparation process for the bone adhesive was the same as in Section 1.3, except that PGA-P was replaced with phosphorylated polyaspartic acid. The adhesive prepared exhibits similar physicochemical properties to the hydrogel prepared with PGA-P: the bone adhesive precursor solution is a flowable, stable, and uniformly dispersed composite, which rapidly cures under the action of the crosslinking agent EDC/NHS.

### Example 2. Rheological Characterization of Injectable Bone Adhesive

To evaluate the injectability of the mixture for preparing bone adhesive, this example employed a rotational rheometer to examine the evolution of storage modulus (G′), loss modulus (G″), and curing time during the adhesive's cure process. The measurements were conducted at 37° C. with fixed parameters (frequency: 10 Hz; strain: 5%; duration: 3 min). The time-dependent curves of G′ and G″ were recorded, and the curing time was calculated accordingly.

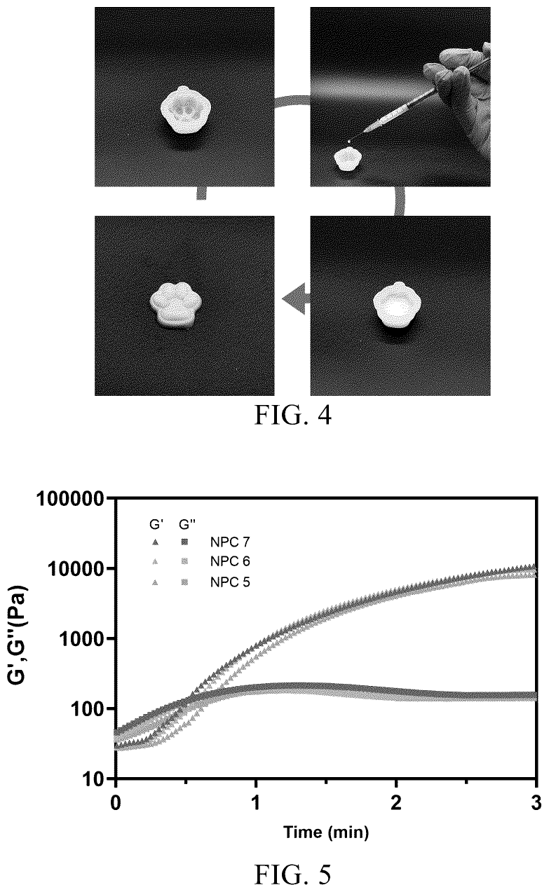

As shown in FIG. 4, the injectable bone adhesive exhibited excellent injectability and topological adaptability, enabling the fabrication of complex shapes. Rheological results (FIG. 5) demonstrated that all hydrogel groups achieved gelation within 0.3-1 min, confirming their superior injectability.

### Example 3. Characterization of Bone-Adhesive Properties of Injectable Bone Adhesive

To evaluate the adhesion of injectable bone adhesives to bone, this study utilized porcine femurs as model bone tissue. Following a lap-shear model, the adhesive was injected between two bone fragments, and bonding strength was measured using a universal testing machine at a crosshead speed of 2 mm/min.

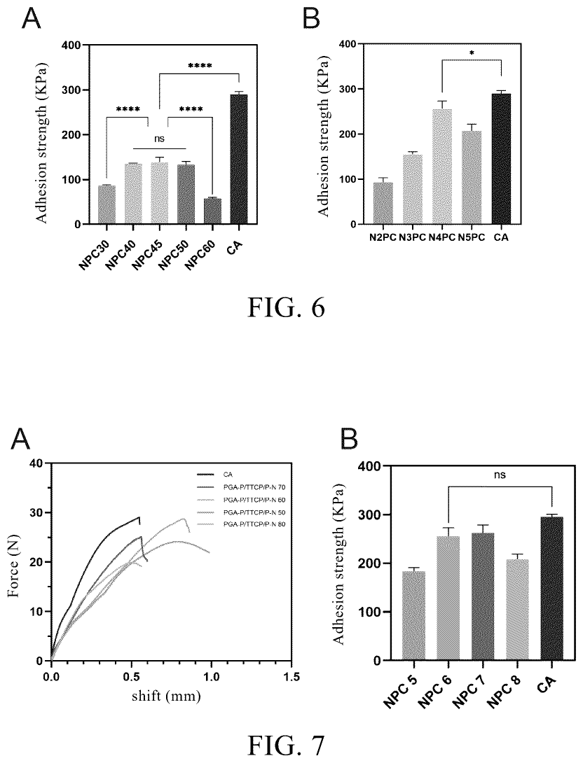

The results are shown in FIGS. 6-7. As can be seen from FIG. 6, by controlling variables, the bone adhesion strength first increased and then decreased with the increase of calcium phosphate salt content and the second polymer content, reaching a peak value. This allowed screening of the material composition with optimal adhesion strength, i.e., the optimal ratio between the first polymer, second polymer and calcium phosphate salt.

As shown in FIG. 7, panel B, the bone adhesive demonstrated excellent bone adhesion strength comparable to 502 glue, meeting the requirements for bone fragment fixation in fracture repair surgery. The bone adhesion strength first increased and then decreased with increasing crosslinking degree, showing a peak value.

### Example 4. Compressive Property Characterization of Injectable Bone Adhesive

To evaluate the compressive properties of the injectable bone adhesive, this study conducted compression tests using a universal testing machine. The compression speed was fixed at 2 mm·min⁻¹, with data recorded to generate compression curves for analyzing the compressive modulus and compression strength of the adhesive.

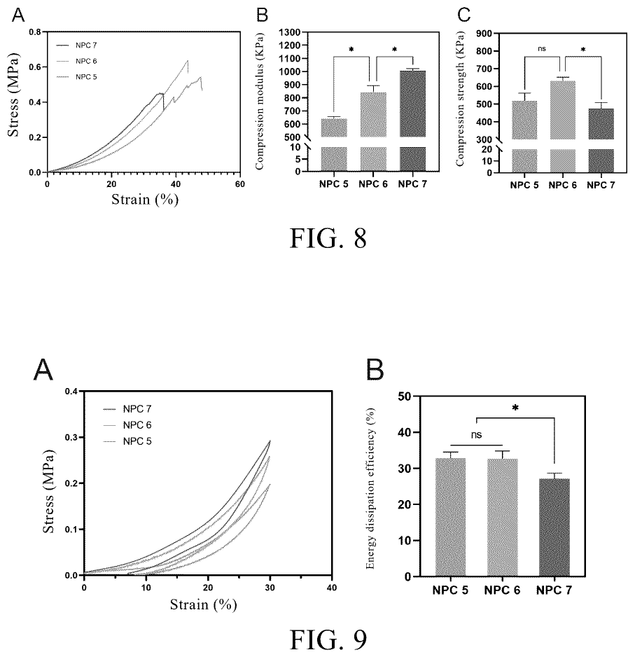

The results are shown in FIG. 8. As seen in FIG. 8, panel B, the compressive modulus of the bone adhesive reached 600-1200 kPa, indicating its high modulus, which ensures minimal shift under potential load-bearing conditions in practical applications. The compressive modulus increased with higher crosslinking degrees. From FIG. 8, panel C, the compressive strength of the bone adhesive achieved 400-700 kPa, demonstrating that the bone adhesive has high compressive strength and is resistant to damage. The compressive strength first increases and then decreases with increasing cross-linking degree, reaching a peak.

### Example 5. Cyclic Loading Compression Characterization of Injectable Bone Adhesive

To evaluate the energy dissipation and fatigue resistance properties of the injectable bone adhesive, cyclic compression tests were performed using a universal testing machine. The tests were conducted at a constant compression rate of 2 mm/min, applying 30% strain followed by stress release to 0 MPa, with 10 loading-unloading cycles. The resulting cyclic compression curves were analyzed to determine the adhesive's energy dissipation efficiency and fatigue resistance.

The results are presented in FIG. 9. As shown in FIG. 9, panel B, the bone adhesive exhibits notable energy dissipation characteristics, ensuring that energy dissipation occurs when subjected to impact energy in actual application, thereby reducing damage to the material itself. The energy dissipation efficiency first increases and then decreases with the increase of crosslinking degree, reaching a peak.

### Example 6. Mechanism Investigation of Injectable Bone Adhesive

To evaluate the effects of chemical crosslinking versus physical crosslinking on material properties, each adhesive group was immersed in ultrapure water for 24 hours after curing. Excess water was then removed to examine swelling behavior and compressive performance.

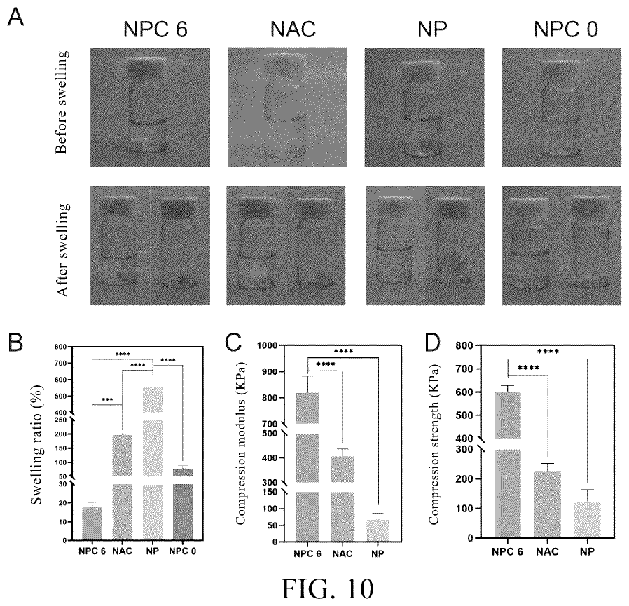

The results are shown in FIG. 10. From FIGS. 10, panels A and B, formulations without calcium phosphate salts exhibited significant water absorption and swelling, whereas those containing calcium phosphate salts effectively controlled swelling through electrostatic interactions between negatively charged groups and calcium ions. Phosphorylated PGA showed reduced swelling compared to unmodified PGA, indicating stronger electrostatic interactions between phosphate groups and calcium ions than carboxyl groups. From FIGS. 10, panels C and D, the compression strength and modulus of the injectable bone adhesive were significantly improved after loading with calcium phosphate salts. Further enhancement of compression strength was observed with phosphorylated PGA modification, confirming that the electrostatic interaction between phosphate groups and calcium ions is stronger than that between carboxyls, thereby improving material cohesion. The non-chemically crosslinked NPC 0 group showed minimal swelling but negligible compressive properties that could not be measured by the instrument.

### Example 7. Cell Compatibility Characterization of Injectable Bone Adhesive

To evaluate the cell compatibility of the injectable bone adhesive, this experiment examined the cytotoxicity at 1, 4, and 7 days through co-culture with bone marrow mesenchymal stem cells (BMSCs). The specific protocol was as follows: first, the bone adhesive was prepared in situ and sterilized at the bottom of 96-well plates; then, cells were seeded on the adhesive surface at a density of 5×10³ cells per well; finally, cell viability was assessed using CCK-8 assay at 1, 4, and 7 days. As shown in the figures, the NPC group (NPC6) was selected as the optimal material formulation and served as the control for subsequent cellular and animal experiments. The CA group represented clinically used cyanoacrylate-based tissue adhesives, which are known to have high adhesive strength but suffer from cytotoxicity and poor degradability.

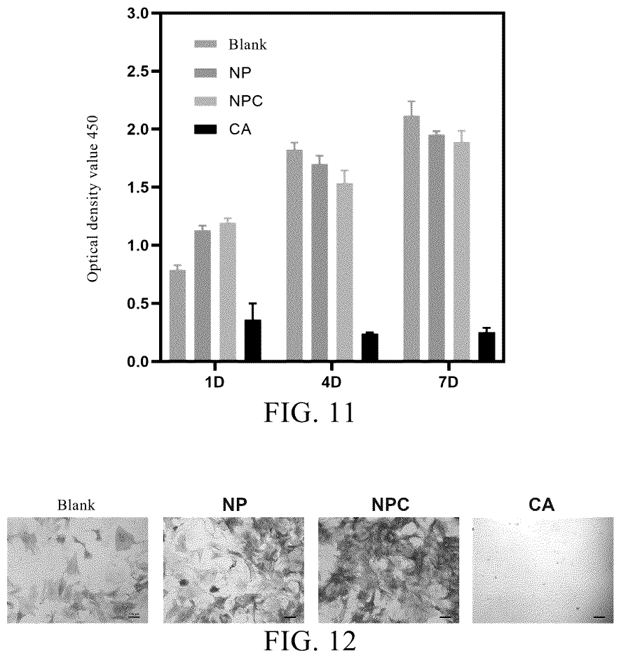

FIG. 11 demonstrates that all bone adhesive groups showed no significant cytotoxicity toward BMSCs compared to the tissue culture plate (TCP) control after 1, 4, and 7 days of co-culture, indicating that the injectable bone adhesive described in this invention exhibits excellent cytocompatibility.

### Example 8. Characterization of Osteogenic Properties of Injectable Bone Adhesive

To evaluate the in vitro osteogenic properties of the injectable bone adhesive, this study selected NP and NPC6 as material groups and examined alkaline phosphatase (ALP) activity and mineralization after 2 weeks of co-culture with bone marrow mesenchymal stem cells (BMSCs).

The specific protocol was as follows: samples were prepared according to the aforementioned grouping method. BMSCs were seeded at a density of 5×10³ cells/well in 24-well plates and cultured in commercial osteogenic induction medium (RAXMX-90021, OriCell®), which contained α-MEM supplemented with 2% FBS, 0.05 mM L-ascorbic acid, 100 mM β-glycerophosphate, and 100 mM dexamethasone. After 2 weeks of culture, remove the culture medium, wash twice with PBS, stain the cells using the BCIP/NBT alkaline phosphatase color development kit, and take photographs.

As shown in FIG. 12, the injectable bone adhesive described in this invention exhibits good in vitro osteogenic properties.

### Example 9. In Vivo Fracture Adhesion and Repair Characterization of Injectable Bone Adhesive

To evaluate the in vivo fracture adhesion and repair performance of the injectable bone adhesive, this study utilized NPC6 as the material group and rat tibiae as animal models. A 1 cm long bone fragment was created by cutting along one-third thickness of the tibial using a reciprocating saw, followed by injection of the bone adhesive to bond the fragments. After gelation, the wound was sutured. and observations were conducted at 4 and 8 weeks post-surgery.

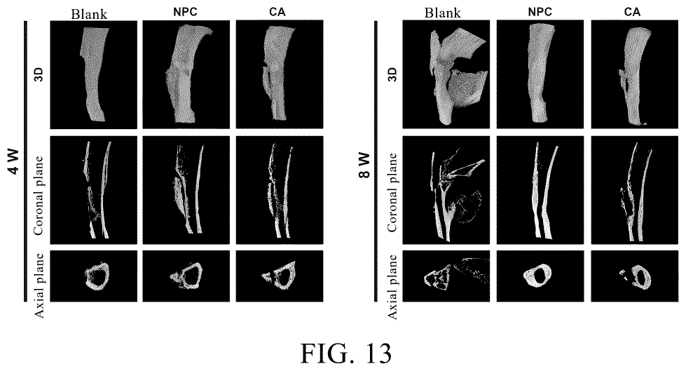

As shown in FIG. 13, the injectable bone adhesive described in this invention demonstrated excellent in vivo fracture adhesion and repair performance. The adhesive maintained strong adhesion at 4 weeks post-repair, and by 8 weeks, the overall healing was satisfactory with no distinct interface observed between the bone fragment and the main tibial structure.

### Discussion

In this invention, the inventors have developed—through extensive and in-depth research integrating bone tissue characteristics with clinical needs for fracture repair—a novel injectable bone adhesive that addresses the shortcomings of reported adhesives (e.g., poor mechanical/adhesive strength, inadequate degradation hindering osteogenesis and fracture healing). This novel injectable bone adhesive combines high mechanical/adhesive strength with osteogenic potential for bonding bone fragment in fracture repair surgery.

The present invention employs amino-functionalized polyhydroxy-polyethylene glycolylated polyglyceryl sebacate (PEGS-NH₂) and phosphorylated poly-γ-glutamic acid (PGA-P) as the primary matrix materials, supplemented with tetracalcium phosphate (TTCP). The bone adhesive precursor solution with a certain viscosity is obtained via electrostatic interactions between phosphate/carboxyl groups and calcium ions. Finally, under the action of the crosslinking agent EDC/NHS, the above-mentioned bone adhesive precursor solution can be mixed and injected onto the surface of bone tissue, where it rapidly cures in situ and forms a strong adhesive bond. In addition, the hydrogel also exhibits excellent mechanical properties and promotes osteogenesis.

Specifically, the injectable bone adhesive of the present invention has a double cross-linked network internally, including covalent chemical cross-linking formed by PEGS-NH₂ and PGA-P in the organic component under the action of a cross-linking agent, and electrostatic physical cross-linking spontaneously formed between PGA-P and inorganic calcium phosphate salts. Additionally, the injectable bone adhesive of the present invention can form various bonds with the surface of bone tissue, including stable amide covalent bonds, electrostatic chelation of phosphate groups and carboxyl groups with calcium ions, and hydrogen bonding. The introduction of calcium phosphate salt components not only enhances the mechanical properties of the bone adhesive but also improves its osteoconductivity, promoting new bone regeneration and facilitating fracture healing. Furthermore, it exhibits excellent injectability, enabling it to be injected onto the bone tissue surface, where it cures in situ to form a gel and establishes strong adhesive bonds.

In summary, the injectable bone adhesive of the present invention has a suitable curing time, good injectability, excellent mechanical and adhesive properties, good fatigue resistance, and a certain bone-promoting effect. Consequently, the injectable bone adhesive of this invention is suitable for bonding bone fragments in fracture repair surgeries, particularly for comminuted fractures, while simultaneously promoting bone regeneration. It represents a promising injectable bone adhesive material with significant clinical application potential.

All documents cited in this invention are incorporated herein by reference as if each document were cited individually as a reference. It is further to be understood that after reading the foregoing teachings of the present invention, a person skilled in the art may make various alterations or modifications to the present invention, and these equivalent forms are also within the scope of the appended claims.

1. An injectable bone adhesive, wherein the injectable bone adhesive comprises the following components:
(c1) an organic component, wherein the organic component includes: a first polymer and a second polymer;
(c2) an inorganic component, which the inorganic component includes calcium phosphate salt and derivative thereof.

2. The injectable bone adhesive according to claim 1, wherein the first polymer is a modified polyester polymer, selected from the group consisting of: amino-functionalized polyhydroxyl polyethylene glycolylated polyglyceryl sebacate PEGS-NH₂, amino-functionalized polyglyceryl sebacate, and amino-functionalized polyethylene glycolylated polyglyceryl sebacate.

3. The injectable bone adhesive according to claim 1, wherein the second polymer is polyamino acid or phosphorylated derivative thereof, selected from the group consisting of: polyglutamic acid, phosphorylated polyglutamic acid PGA-P, polyaspartic acid, and phosphorylated polyaspartic acid.

4. The injectable bone adhesive according to claim 1, wherein the mass ratio of the first polymer to the second polymer in the organic component is 2:1 to 1:3.

5. The injectable bone adhesive according to claim 1, wherein the calcium phosphate salt and derivatives thereof are selected from the group consisting of: hydroxyapatite, tetracalcium phosphate, tricalcium phosphate, octacalcium phosphate, and calcium dihydrogen phosphate, either alone or in combination of two or more.

6. The injectable bone adhesive according to claim 1, wherein the mass ratio of the organic component to the inorganic component is 1:1 to 4:1.

7. A method for preparing the injectable bone adhesive according to claim 1, wherein the method comprising the steps of:
(a) providing a bone adhesive precursor solution, wherein the bone adhesive precursor solution comprises a first polymer, a second polymer, and calcium phosphate salt;
(b) providing a crosslinking agent solution, wherein the crosslinking agent comprises 1-(3-dimethylaminopropyl)-3-ethylcarbodiimide hydrochloride (EDC) and N-hydroxy succinimide (NHS);
(c) mixing the bone adhesive precursor solution with the crosslinking agent solution to obtain the injectable bone adhesive,
wherein the preparation of the bone adhesive precursor solution includes the following steps:
(a1) dissolving the first polymer and the second polymer in a solvent to obtain a mixed solution;
(a2) adding the calcium phosphate salt to the above mixed solution to obtain the bone adhesive precursor solution.

8. The method according to claim 7, wherein the crosslinking agent in step (b) is 1-(3-dimethylaminopropyl)-3-ethylcarbodiimide hydrochloride (EDC) and N-hydroxy succinimide (NHS) for catalyzing the formation of amide bond.

9. The method according to claim 7, wherein the molar ratio of EDC to NHS in the crosslinking agent in step (b) is 0.5-2:1, and the molar ratio of EDC to the amino groups in the first polymer is 0.5-1:1.

10. A method for treating fracture comprising administering to a subject in need thereof the injectable bone adhesive according to claim 1 to bond bone fragments, fill bone defects or guide tissue regeneration.

11. The method according to claim 10, wherein the fracture is comminuted fracture.
